(19) United States
(12) Patent Application Publication
(10) Pub. No.: US 2017/0100635 A1
(43) Pub. Date: Apr. 13, 2017

(54) EXERCISE SUGGESTION DEVICE, EXERCISE SUGGESTION METHOD, AND EXERCISE SUGGESTION PROGRAM

(71) Applicant: SEIKO EPSON CORPORATION, Tokyo (JP)

(72) Inventors: Jun TAKEUCHI, Matsumoto-shi (JP); Junko HASEGAWA, Koganei-shi (JP); Junichi OTSUKA, Chikuma-shi (JP)

(73) Assignee: SEIKO EPSON CORPORATION, Tokyo (JP)

(21) Appl. No.: 15/128,816

(22) PCT Filed: Mar. 11, 2015
(86) PCT No.: PCT/JP2015/001350

§ 371 Date: Sep. 23, 2016

(30) Foreign Application Priority Data
Mar. 24, 2014 (JP) 2014-060719

(51) Int. Cl. A63B 24/00; A61B 5/11; A61B 5/145; A61B 5/22; A63B 71/06; A61B 5/00

(52) U.S. Cl. CPC A63B 24/0075; A63B 71/0686; A63B 71/0622; A61B 5/486; A61B 5/4866; A61B 5/14532; A61B 5/22; A61B 5/0024; A61B 5/1118; A63B 2230/203

(57) ABSTRACT

An exercise suggestion device, an exercise suggestion method and an exercise suggestion program that can suggest an effective exercise are to be provided.

An exercise suggestion device includes: an exercise correction unit which corrects exercise information set on the basis of user information about a user and including exercise duration and exercise intensity of an exercise to be carried out by the user, on the basis of dietary information indicating composition of a meal of the user; and a suggestion unit which suggests at least one of the exercise intensity included in the exercise information that is corrected and an exercise type corresponding to the exercise intensity, and the exercise duration included in the exercise information that is corrected.

This application claims the benefit of Japanese Patent Application No. 2014-060719, filed on Mar. 24, 2014. The content of the aforementioned application is incorporated herein by reference in its entirety.

### TECHNICAL FIELD

The present invention relates to an exercise suggestion device, an exercise suggestion method and an exercise suggestion program.

### BACKGROUND ART

Traditionally, a blood sugar level data analysis system which analyzes a blood sugar level data of a diabetic or a person suspected of having diabetes and thus finds an indicator of the glucose metabolism function is known (see PTL 1, for example).

This blood sugar level data analysis system disclosed in PTL 1 measures the blood sugar level of a subject (a patient or person having a medical consultation, or the like) and finds a plurality of parameters as indicators of the glucose metabolism function, using various calculation formulas based on the measurement data. Also, this analysis system can determine whether the subject has diabetes or is on the borderline (between diabetic and normal), by referring to the result of determination based on the comparison between each parameter and its corresponding threshold.

### CITATION LIST

### Patent Literature

PTL 1: Japanese Patent No. 4728616

### SUMMARY OF INVENTION

### Technical Problem

By the way, as treatments for lifestyle diseases such as diabetes, dietary therapies, exercise therapies and pharmacotherapies are known. Of these, dietary therapies and exercise therapies are very important treatments, which are broadly adopted not only for diabetics but also for people likely to develop diabetes (people determined as being on the borderline).

However, in the case of dietary therapies and exercise therapies, it is common that guidance is given to each individual. Therefore, there is a possibility that diabetics and people likely to develop diabetes are not given guidance on exercises in consideration of their dietary composition and therefore cannot carry out effective exercises.

An object of the invention is to provide an exercise suggestion device, an exercise suggestion method and an exercise suggestion program that enable suggestion of effective exercises.

### Solution to Problem

An exercise suggestion device according to a first aspect of the invention includes: an exercise correction unit which corrects exercise information set on the basis of user information about a user and including exercise duration and exercise intensity of an exercise to be carried out by the user, on the basis of dietary information indicating composition of a meal of the user; and a suggestion unit which suggests at least one of the exercise intensity included in the exercise information that is corrected and an exercise type corresponding to the exercise intensity, and the exercise duration included in the exercise information that is corrected.

As the user information, the age of the user or the presence/absence of exercise habits can be given as an example, and changes in the blood sugar level or the like that can be measured on the basis of the blood sugar level in normal time, OGTT (oral glucose tolerance test), and maximum and resting heart rates (maximum and resting pulse rates) or the like, can also be given as an example. Also, the exercise information based on the user information may be set by the exercise suggestion device on the basis of the user information, or may be set by the user of the exercise suggestion device or by a medical practitioner or the like. Moreover, the exercise type indicates a specific type of exercise and can be walking, running, cycling, and swimming or the like, for example.

According to the first aspect, the exercise information set on the basis of the user information is corrected by the exercise correction unit on the basis of the dietary information about a meal which the user has had. Then, in addition to the exercise duration included in the corrected exercise information, the exercise intensity included in this exercise information and the exercise type corresponding to this exercise intensity are suggested by the suggestion unit. Thus, an exercise corresponding to the composition of the meal of the user can be suggested to this user. Therefore, for example, an exercise which can effectively restrain a rise in the blood sugar level after the meal can be suggested to the user. Also, when the exercise type corresponding to the exercise intensity is suggested, the user can more easily grasp the exercise that should be actually carried out.

In the first aspect, it is preferable that the exercise correction unit corrects the exercise information on the basis of an amount of glucide in the meal included in the dietary information.

Here, if the amount of glucide in the meal is large, the maximum blood sugar level after the meal tends to be higher than in the case where the amount of glucide is small.

Therefore, according to the first aspect, since the exercise information is corrected according to the amount of glucide in the meal, an exercise that can effectively restrain a rise in the blood sugar level after the meal can be securely suggested to the user.

In the first aspect, it is preferable that the exercise correction unit corrects the exercise information on the basis of a ratio of protein, fat and carbohydrate in the meal included in the dietary information.

Here, the way the blood sugar level rises changes, depending on the ratio of protein, fat and carbohydrate (so-called PFC ratio) in the meal. For example, if the proportion of protein in the meal is high, the maximum blood sugar level after the meal tends to be low, but the period during which the maximum blood sugar level is maintained (balanced phase) tends to be long. If the proportion of fat in the meal is high, this tendency is more prominent. Meanwhile, if the proportion of carbohydrate in the meal is high, the maximum blood sugar level after the meal tends to be high, but the period during which the maximum blood sugar level is maintained tends to be short.

Therefore, in the first embodiment, since the exercise information is corrected on the basis of the ratio of protein, fat and carbohydrate in the meal, an exercise that can more effectively restrain a rise in the blood sugar level after the meal can be securely suggested to the user.

In the first aspect, it is preferable that the device includes a timing setting unit which sets a start timing of the exercise on the basis of blood sugar level change information indicating the way the blood sugar level of the user included in the user information rises, and that the suggestion unit suggests the start timing.

Here, if the start timing of the exercise is set in a period during which the blood sugar level rises after the meal (ascent phase), the maximum blood sugar level can be reduced by carrying out the exercise, compared with the case where the exercise is not carried out. Meanwhile, if the start timing of the exercise is set in a period during which the maximum blood sugar level is maintained (balanced phase) or set in a period during which the blood sugar level falls (descent phase), the blood sugar level can be lowered relatively easily even by a low-intensity exercise.

Therefore, in the first aspect, by setting the start timing according to the user's objective, an exercise corresponding to the objective can be suggested.

If the start timing of the exercise is set in the ascent phase, since the maximum blood sugar level can be reduced as described above by carrying out the exercise, the range of fluctuation in the blood sugar level can be reduced. Therefore, damage to blood vessels due to the fluctuation in the blood sugar level can be restrained. Also, improvement in insulin resistance is expected if the exercise is executed repeatedly. Thus, it is preferable that the start timing of the exercise is set in a period before the blood sugar level reaches the maximum blood sugar level.

Meanwhile, since the ascent period of the blood sugar level varies depending on the PFC ratio in the meal, as described above, the start timing may be set in consideration of the dietary composition.

In the first aspect, it is preferable that the device includes a post-exercise blood sugar level prediction unit which predicts a post-exercise blood sugar level change that is a change in the blood sugar level of the user in the case where the exercise indicated by the exercise information is carried out, and that the suggestion unit suggests the post-exercise blood sugar level change. In the first aspect, since the post-exercise blood sugar level change predicted by the post-exercise blood sugar level prediction unit is suggested, the user can grasp how the blood sugar level changes as the user carries out the suggested exercise. Therefore, the user's motivation for the exercise can be enhanced.

In the first aspect, it is preferable that the device includes a post-meal blood sugar level prediction unit which predicts a post-meal blood sugar level change that is a change in the blood sugar level of the user in the case where the meal is taken, on the basis of the dietary information, and the suggestion unit suggests the post-meal blood sugar level change and the post-exercise blood sugar level change.

According to the first aspect, the post-meal blood sugar level change predicted by the post-meal blood sugar level prediction unit is suggested in addition to the post-exercise blood sugar level change. Thus, the user can compare changes in the blood sugar level between the case where the suggested exercise is carried out and the case where the suggested exercise is not carried out. Therefore, the user's motivation for the exercise can be enhanced further.

An exercise suggestion method according to a second aspect of the invention is an exercise suggestion method carried out using an exercise suggestion device which suggests details of an exercise to be carried out by a user. The method includes: correcting exercise information set on the basis of user information about the user and including exercise duration and exercise intensity of an exercise to be carried out by the user, on the basis of dietary information indicating composition of a meal of the user; and suggesting at least one of the exercise intensity included in the exercise information that is corrected and an exercise type corresponding to the exercise intensity, and the exercise duration included in the exercise information that is corrected.

According to the second aspect, effects similar to those of the exercise suggestion device according to the first aspect can be achieved by applying the exercise suggestion method to the exercise suggestion device.

An exercise suggestion program according to a third aspect of the invention is an exercise suggestion program executed by an exercise suggestion device which suggests details of an exercise to be carried out by a user. The program causes the exercise suggestion device to execute: correcting exercise information set on the basis of user information about the user and including exercise duration and exercise intensity of an exercise to be carried out by the user, on the basis of dietary information indicating composition of a meal of the user; and suggesting at least one of the exercise intensity included in the exercise information that is corrected and an exercise type corresponding to the exercise intensity, and the exercise duration included in the exercise information that is corrected.

According to the third aspect, effects similar to those of the exercise suggestion device according to the first aspect can be achieved by applying the exercise suggestion program to the exercise suggestion device.

Such an exercise suggestion program may be recorded on a computer-readable recording medium. As the exercise suggestion device reads and executes the exercise suggestion program from such a recording medium, effects similar to those of the exercise suggestion device according to the first aspect can be achieved. As such a recording medium, a magnetic tape, magnetic disk, optical disk, magneto-optical disk, HDD (hard disk drive) and semiconductor memory or the like can be adopted as an example. Using these, the exercise suggestion program can be executed at an information processing device. Moreover, this exercise suggestion program may also be provided via a network.

### BRIEF DESCRIPTION OF DRAWINGS

FIG. 1 is a schematic view showing an exercise support system according to an embodiment of the invention.

FIG. 2 is a block diagram showing the configuration of an information processing device in the embodiment.

FIG. 3 is a block diagram showing the configuration of a control unit in the embodiment.

FIG. 4 is a view showing an example of a menu screen in the embodiment.

FIG. 5 is a view showing an example of an exercise display screen in the embodiment.

FIG. 6 is a view showing an example of a physical information registration screen in the embodiment.

FIG. 7 is a view showing an example of a dietary composition registration screen in the embodiment.

FIG. 8 is a view showing an example of a simplified registration screen in the embodiment.

FIG. 9 is a view showing an example of a medication status registration screen in the embodiment.

FIG. 10 is a view showing an example of a sleep registration screen in the embodiment.

FIG. 11 is a view showing an example of a calendar display screen in the embodiment.

FIG. 12 is a view showing an example of changes in the blood sugar level corresponding to the degree of progression of diabetes in the embodiment.

FIG. 13 is a view showing an example of changes in the blood sugar level corresponding to PFC ratios in the embodiment.

FIG. 14 is a view showing an example of an exercise suggestion screen in the embodiment.

FIG. 15 is a view showing an example of an information suggestion screen in the embodiment.

FIG. 16 is a flowchart showing exercise suggestion processing in the embodiment.

### DESCRIPTION OF EMBODIMENTS

Hereinafter, an embodiment of the invention will be described on the basis of the drawings.

### [Schematic Configuration of Exercise Support System]

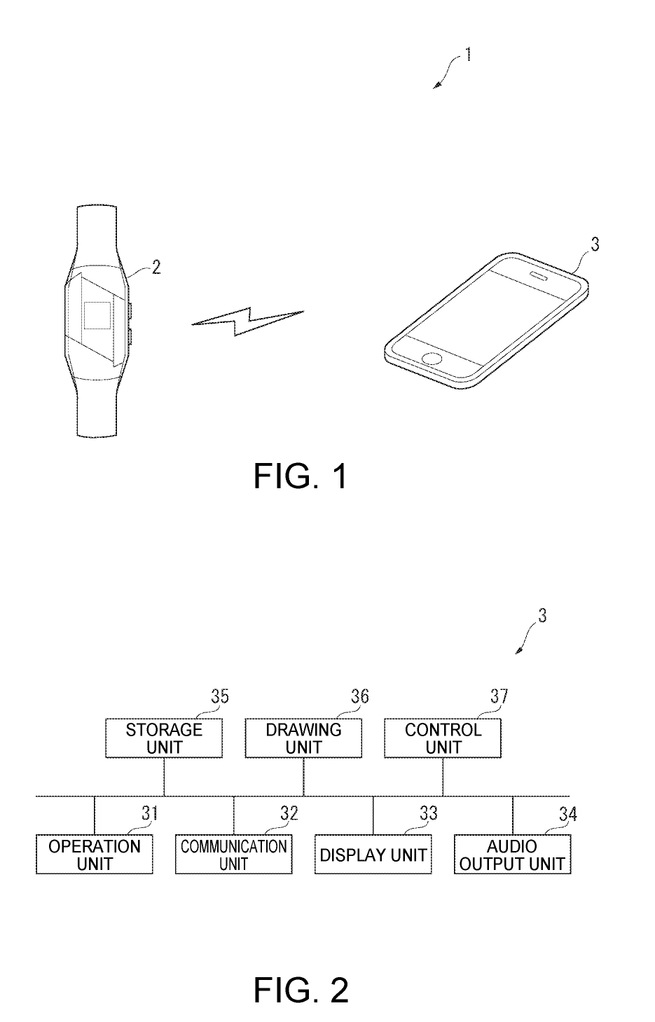

FIG. 1 is a schematic view showing an exercise support system 1 according to this embodiment.

The exercise support system 1 according to this embodiment includes a measuring device 2 and an information processing device 3, as shown in FIG. 1. In this exercise support system 1, the information processing device 3 holds action information about actions of a user measured by the measuring device 2 and also suggests an exercise suitable for the user on the basis of dietary information indicating the dietary composition of the user and user information about the user that are inputted.

### [Configuration of Measuring Device]

The measuring device 2 is configured in such a way as to be able to be mounted on the user and record and transmit the action information of the user. As such a measuring device 2, a pulse meter which measures biological information such as pulse rates of the user and motion information such as acceleration can be given as an example, and an activity monitor which measures the motion information, measures the number of steps taken by the user and thus calculates the calories burned can be given as an example as well.

### [Configuration of Information Processing Device]

FIG. 2 is a block diagram showing the configuration of the information processing device 3.

The information processing device 3 stores the action information received from the measuring device 2. Also, the information processing device 3 is equivalent to the exercise suggestion device of the invention and suggests an exercise suitable for the user (for example, an exercise by which the maximum blood sugar level can be reduced) on the basis of the dietary information or the like inputted by the user. This information processing device 3 can be formed by a smartphone (multifunctional mobile phone), tablet and PC (personal computer) or the like, for example.

Such an information processing device 3 includes an operation unit 31, a communication unit 32, a display unit 33, an audio output unit 34, a storage unit 35, a drawing unit 36, and a control unit 37, as shown in FIG. 2.

The operation unit 31 accepts an input operation from the user and outputs operation information corresponding to this input operation to the control unit 37. Such an operation unit 31 can be formed, for example, by physical keys and a touch panel or the like provided on the casing of the information processing device 3, and can also be formed by a keyboard and pointing device or the like connected to the information processing device 3.

The communication unit 32 has a first communication module capable of communicating with an external device such as the measuring device 2 and a second communication module capable of communicating with a server (not illustrated) on a network such as the Internet, and communicates with the external device and the server under the control of the control unit 37. If it is possible to communicate with each of the measuring device 2 and the server by the same communication method, there has to be only one of the first communication module and the second communication module. If the communication with the server is unnecessary, the second communication module may be omitted.

The display unit 33 displays an image drawn by the drawing unit 36. Specifically, the display unit 33 displays an execution screen for an OS (operating system) and applications executed by the control unit 37. That is, the display unit 33 functions as a suggestion unit which displays an execution screen for an exercise support application, described later, and then suggests exercise information or the like to the user. Such a display unit 33 can be formed, for example, by various display panels such as liquid crystal, organic EL (electro-luminescence) and electrophoretic types.

The audio output unit 34 is configured with a speaker and outputs a sound corresponding to audio information inputted from the control unit 37. For example, when the control unit 37 executes an exercise support application, the audio output unit 34 outputs a sound corresponding to information suggested to the user. That is, the audio output unit 34 is equivalent to the suggestion unit of the invention, similarly to the display unit 33.

The storage unit 35 is formed by an HDD (hard disk drive) and flash memory or the like, and stores a program and data necessary for operations of the information processing device 3. As such a program, for example, the storage unit 35 stores an exercise support application (exercise suggestion program) which causes the control unit 37 to execute exercise suggestion processing, described later, in addition to the OS which controls the information processing device 3. Also, as the data, for example, the storage unit 35 stores user information and also stores the action information received from the measuring device 2 and various kinds of information inputted on the execution screen for the exercise support application. Moreover, the storage unit 35 stores a table where exercise intensities and exercise types such as walking, running, and cycling, are associated with each other.

The drawing unit 36 is formed by a drawing circuit and draws an image to be displayed on the display unit 33, under the control of the control unit 37. For example, the drawing unit 36 draws an execution screen when the control unit 37 executes the exercise suggestion program stored in the storage unit 35.

### [Configuration of Control Unit]

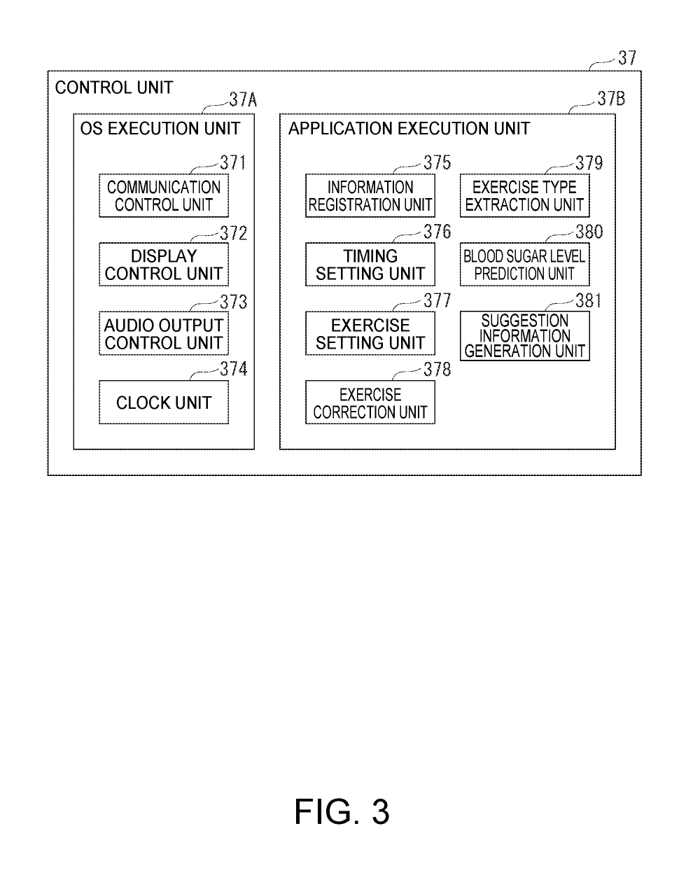

FIG. 3 is a block diagram showing the configuration of the control unit 37. The control unit 37 configured with a CPU (central processing unit) and controls operations of the information processing device 3 by having the CPU execute the program stored in the storage unit 35. Such a control unit 37 includes an OS execution unit 37A and an application execution unit 37B.

The OS execution unit 37A is a functional unit which executes the OS stored in the storage unit 35 and includes a communication control unit 371, a display control unit 372, an audio output control unit 373, and a clock unit 374.

The communication control unit 371 controls the communication unit 32 to communicate with an external device. The display control unit 372 controls the drawing unit 36 and thus causes the drawing unit 36 to draw an execution screen for the OS and applications and causes the display unit 33 to display the execution screen.

The audio output control unit 373 outputs audio information of a sound outputted at the time of executing the OS and applications, to the audio output unit 34.

The clock unit 374 measures the current time.

The application execution unit 37B executes an application designated by the OS execution unit 37A according to operation information inputted from the operation unit 31, of the applications stored in the storage unit 35.

### [Configuration of Execution Screen]

The display control unit 372 causes the drawing unit 36 to draw execution screens ES (ES1 to ES9) described below, and causes the display unit 33 to display these individual screens ES, when the application execution unit 37B executes the exercise support application.

Hereinafter, the configurations and operations of the individual screens ES will be described.

### [Configuration and Operation of Menu Screen]

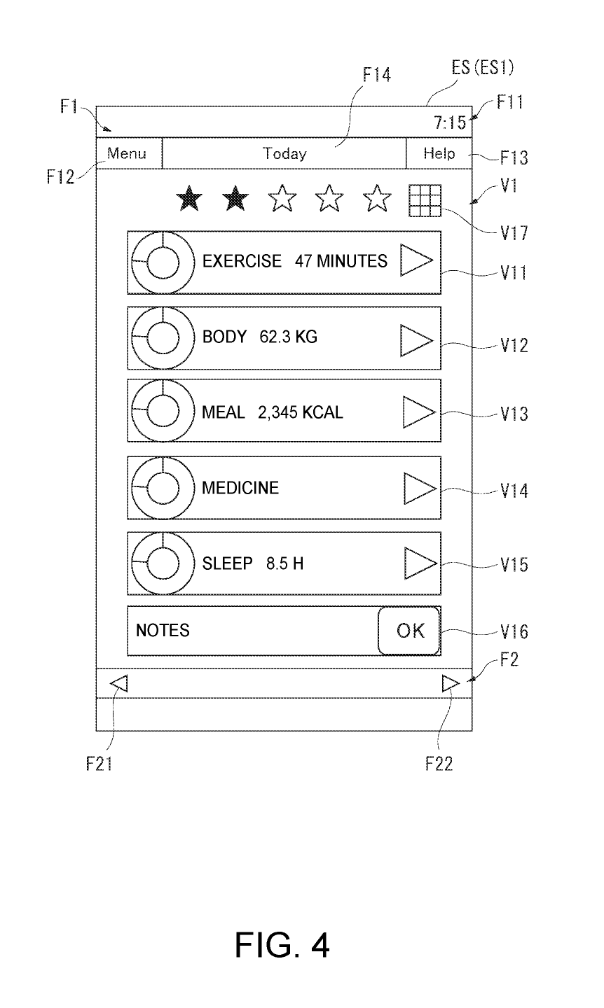

FIG. 4 is a view showing an example of the menu screen ES1, of the execution screens ES displayed at the time of executing the exercise support application. The menu screen ES1 shown in FIG. 4 is a screen displayed after a login is made on a login screen as the exercise support application is executed. In this menu screen ES1, fixed display areas F1, F2 are set at the top of the screen and the bottom of the screen, and a variable display area V1 is set between these.

In the fixed display area F1 at the top of the screen, a time display area F11 where the current time is set is arranged. Also, a button F12 for shifting to this menu screen ES1 when pressed (entered) is arranged on the left side of the fixed display area F1, and a button F13 for shifting to a help screen when pressed is arranged on the right side. Also, a title F14 indicating the details set in the variable display area V1 is arranged substantially at the center of this fixed display area F1.

On the left and right sides of the fixed display area F2 at the bottom of the screen, buttons F21, F22 are arranged. These buttons F21, F22 are buttons to shift screens. In the menu screen ES1, every time the button F21 is pressed, the screen shifts to the menu screen of the previous date, whereas every time the button F22 is pressed, the screen shifts to the menu screen of the later date.

In the variable display area V1, five selection items V11 to V15 are arranged in series in a vertical direction. To these selection items V11 to V15, item names of these selection items V11 to V15 and data related to the selection items V11 to V15 are set. For example, in the selection item V11 set at the top, “exercise” is given as the item name, and “47 minutes”, which is the exercise duration of the day found on the basis of data that is already inputted, is given to the right of this item name. Also, for example, in the selection item V13 set in the middle, “meal” is given as the item name, and “2,345 kcal”, which is the calorie intake of the day found on the basis of dietary information that is already inputted, is given to the right of this item name. The selection items V12, V15 marked with “body” and “sleep” are similarly configured. However, the selection item V14 marked with “medicine” has the item name only.

When one of these selection items V11 to V15 is pressed, the screen shifts to the screen ES2 to ES6 for the corresponding selection item.

Moreover, an input area V16 where notes can be entered is arranged at the bottom of the variable display area V1. The details entered in the input area V16 are stored in the storage unit 35 in association with the date of the menu screen ES1 that is currently displayed. Therefore, when the menu screen ES1 of this date is displayed again, these details can be viewed.

Meanwhile, a lattice-like button V17 is arranged at a top right part of the variable display area V1. When this button V17 is pressed, the screen shifts to a calendar display screen ES7, described later.

### [Configuration and Operation of Exercise Display Screen]

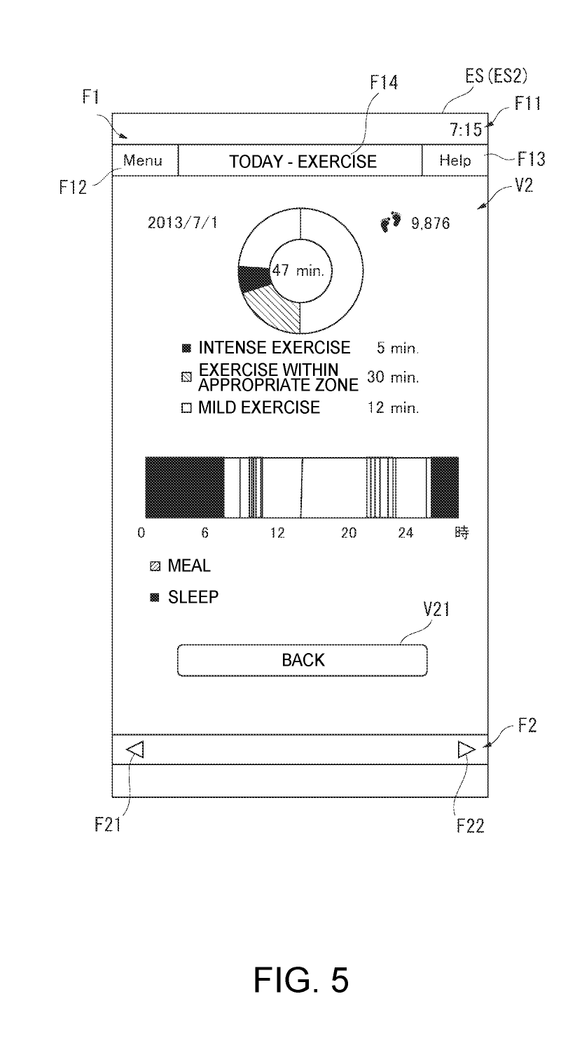

FIG. 5 is a view showing an example of the exercise display screen ES2, of the execution screens ES.

When the selection item V11 is pressed, or when the button F22 is pressed on the sleep registration screen ES6, described later, the exercise display screen ES2 shown in FIG. 5 is displayed. This exercise display screen ES2 is a screen showing the exercise state of the user based on data received from the measuring device 2. This exercise display screen ES2 includes the fixed display areas F1, F2, and a variable display area V2.

The date of displayed data and the number of steps taken by the user corresponding to this date are arranged at the top of the variable display area V2.

Substantially in the middle of the variable display area V2, numeric values and a pie chart showing the level of exercise and exercise duration of the user are arranged, and a chart showing the action type for every hour of that date and the level of exercise, in time series, is arranged as well.

A button V21 marked with “back” is arranged at the bottom of the variable display area V2. Entering this button V21 enables a shift to the menu screen ES1.

When the button F21, of the buttons F21, F22 arranged in the fixed display area F2 of the exercise display screen ES2, is pressed, the screen shifts to the sleep registration screen ES6, described later. When the button F22 is pressed, the screen shifts to a physical information registration screen ES3.

Such an exercise display screen ES2 in this embodiment is configured as a screen showing the exercise state of the user. However, the exercise display screen ES2 is not limited to this and may be formed a screen for the user to input and register the details of his/her own exercise. In this case, the exercise display screen ES2 may be configured in such a way that the user checks whether the exercise suggested as a result of the execution of the exercise suggestion processing, described later, has been carried out or not, thereby storing information about the exercise that has been carried out, in the storage unit 35, or may be configured in such a way that the user can input the start time, exercise duration and exercise intensity or the like of the exercise carries out by the user.

### [Configuration and Operation of Physical Information Registration Screen]

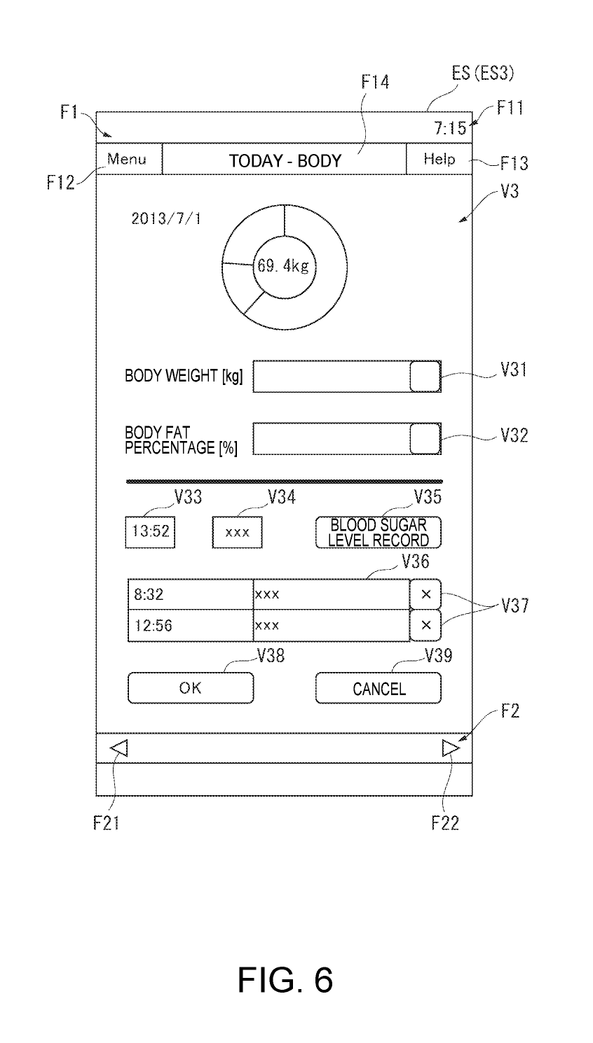

FIG. 6 is a view showing an example of the physical information registration screen ES3, of the execution screens ES.

When the selection item V12 is pressed on the menu screen ES1, or when the button F22 is pressed on the exercise display screen ES2, the physical information registration screen ES3 shown in FIG. 6 is displayed. This physical information registration screen ES3 is a screen for registering and showing physical information of the user, and includes the fixed display areas F1, F2 and a variable display area V3.

At the top of the variable display area V3, the date of displayed data, and a pie chart showing the ratio of the current body fat percentage to a target body fat percentage of the user are arranged.

Substantially in the middle of the variable display area V3, a body weight input section 31, a body fat percentage input section V32, a blood sugar level measuring time input section V33, a blood sugar level input section V34, and a recording button V35 marked with “blood sugar level record” are arranged. Moreover, a table V36 having rows where the measuring time and blood sugar level that are already registered are set, and delete buttons V37 corresponding to these rows are arranged. Below these, a registration button V38 marked with “OK”, and a cancel button V39 marked with “cancel” are arranged. When the registration button V38 is pressed after the body weight, body fat percentage, time and blood sugar level are inputted in the respective input sections V31 to V34, the input details are finalized and these input details are stored in the storage unit 35 and thus registered. When the cancel button V39 is pressed, the screen returns to the menu screen ES1.

Also, when the button F21, of the buttons F21, F22 arranged in the fixed display area F2 of the physical information registration screen ES3, is pressed, the screen shifts to the dietary composition registration screen ES4. When the button F22 is pressed, the screen shifts to the exercise display screen ES2.

### [Configuration and Operation of Dietary Composition Registration Screen]

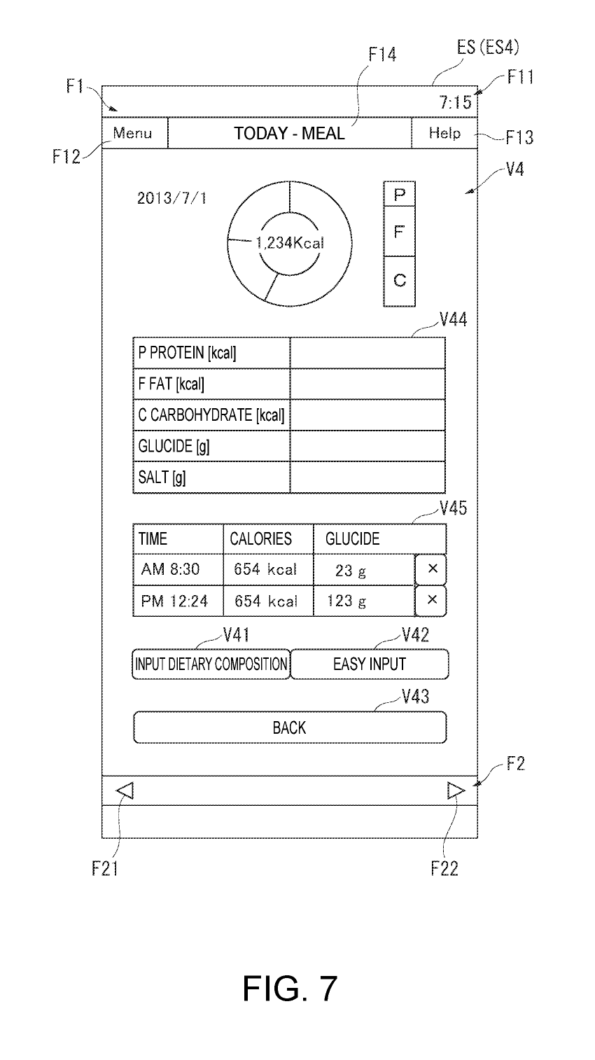

FIG. 7 is a view showing an example of the dietary composition registration screen ES4, of the execution screens ES.

When the selection item V13 is pressed on the menu screen ES1 or when the button F22 is pressed on the physical information registration screen ES3, the dietary composition registration screen ES4 shown in FIG. 7 is displayed. This dietary composition registration screen ES4 is a screen for registering and displaying the composition of a meal which the user has had, and includes the fixed display areas F1, F2 and a variable display area V4.

At the top of the variable display area V4, the date of displayed data, and a pie chart showing the ratio of protein (P), fat (F) and carbohydrate (C) of the calorie intake in one day up to the current time point to a target calorie intake in one day of the user are arranged.

At the bottom of the variable display area V4, a detail registration button V41 marked with “input dietary composition”, a simplified registration button V42 marked with “easy input”, and a back button V43 marked with “back” are arranged.

Of these, when the detail registration button V41 is pressed, a screen where a meal menu and meal volume which the user has had can be selected is displayed, though not illustrated. Then, as the user selects and inputs an item corresponding to the dietary composition on this screen, the dietary composition is stored in the storage unit 35 and thus registered. Also, the dietary composition is analyzed and the calories and ratio of protein, fat and carbohydrate (PFC ratio) in the meal, the amount of glucide, the amount of salt, and the total calories of the meal are calculated (these are collectively referred to as dietary information). The results of the calculations are reflected on a table V44 arranged substantially in the middle of the variable display area V4, and the time of the meal, the total calories and the amount of glucide are reflected on a table V45. Also, the analysis of the dietary composition may be executed by referring to or via an external database.

In rows in the table V45, the time information, the total calories and the amount of glucide are set, of the dietary information that is already registered. When the user selects a row where dietary information is set, details of the dietary information are displayed in the table V44. The time information of the meal may be the start time of the meal, the end time of the meal, or the time taken for the meal.

### [Configuration and Operation of Simplified Registration Screen]

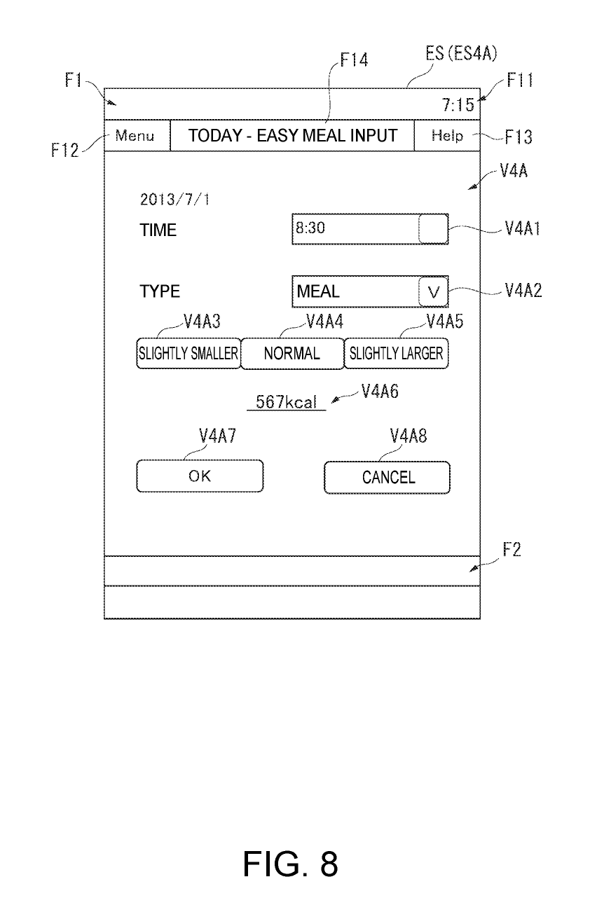

FIG. 8 is a view showing an example of a simplified registration screen ES4A, of the execution screens ES.

Meanwhile, when the simplified registration button V42 is pressed, the screen shifts to the simplified registration screen ES4A shown in FIG. 8. This simplified registration screen ES4A is a screen for registering the time of the meal, the type and the meal volume (subjective information of the meal volume, and relative information), and includes the fixed display areas F1, F2 and a variable display area V4A.

Although the buttons F21, F22 are not arranged in the fixed display area F2 of this simplified registration screen ES4A, the button F21 may be arranged as a button to shift to the physical information registration screen ES3, and the button F22 may be arranged as a button to shift to the medication status registration screen ES5.

In the variable display area V4A, a time input section V4A1 where the time of a meal is inputted, a type selection section V4A2 where a type such as “meal”, “drinking alcohol”, “meal and drinking alcohol” or the like is selected, and meal volume setting buttons V4A3 to V4A5 to be pressed according to the meal volume are arranged. Of these, the button V4A3 is marked with “slightly smaller”, the button V4A4 is marked with “normal”, and the button V4A5 is marked with “slightly larger”. When one of these buttons V4A3 to V4A5 is pressed, the meal volume corresponding to the pressed button is set, and the calorie value calculated on the basis of the type selected in the type selection section V4A2 is displayed in a calorie display area V4A6 arranged substantially in the middle of the variable display area V4A.

Then, when a registration button V4A7 arranged at the bottom of the variable display area V4A is pressed, the dietary composition inputted and selected in the simplified registration screen ES4A is stored in the storage unit 35 and thus registered. Meanwhile, when a cancel button V4A8, similarly arranged at the bottom, is pressed, the screen shifts to the dietary composition registration screen ES4.

As the dietary composition is registered in this manner, the registered composition is reflected on the table V45 in the dietary composition registration screen ES4 shown in FIG. 7.

When the button F21, of the buttons F21, F22 arranged in the fixed display area F2 of the dietary composition registration screen ES4, is pressed, the screen shifts to the medication status registration screen ES5. When the button F22 is pressed, the screen shifts to the physical information registration screen ES3.

### [Configuration and Operation of Medication Status Registration Screen]

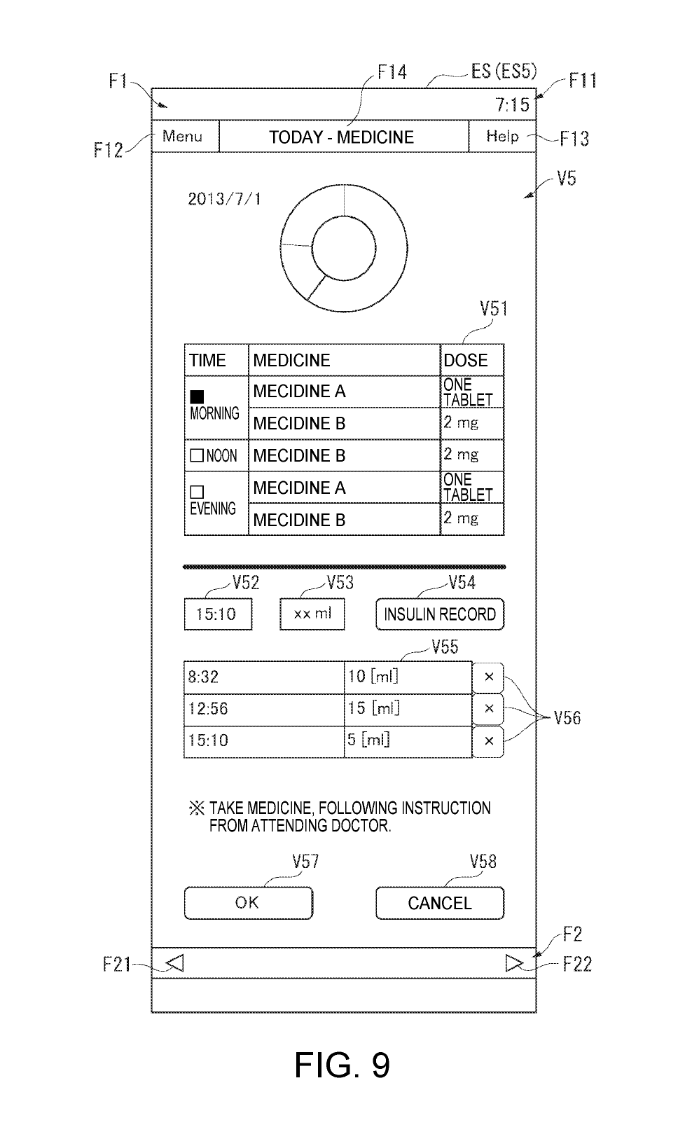

FIG. 9 is a view showing an example of the medication status registration screen ES5, of the execution screens ES.

When the selection item V14 is pressed on the menu screen ES1 or when the button F22 is pressed on the dietary composition registration screen ES4, the medication status registration screen ES5 shown in FIG. 9 is displayed. This medication status registration screen ES5 is a screen for the user to register the details of a medicine which the user has self-administered, and includes the fixed display areas Fl, F2 and a variable display area V5.

At the top of the variable display area V5, the date of displayed data, and a table V51 having set columns where types and doses of medicines to be taken in the morning, at noon and in the evening are set and columns where whether the medicine has been taken or not can be checked, are arranged.

Substantially in the middle of the variable display area V5, input sections V52, V53 where the time when insulin is administered and its unit are inputted, and a recording button V54 marked with “insulin record”, are arranged.

At the bottom of the variable display area V5, a table V55 having rows where the time of administration and insulin unit that are already registered are set, and delete buttons V56 corresponding to these rows are arranged. Further below these, a registration button V57 marked with “OK” and a cancel button V58 marked with “cancel” are set. Then, when the recording button V54 is pressed after the time of administration and insulin unit are inputted in the respective input sections V52, V53, the inputted details are reflected on the table V55. Meanwhile, when the delete button V56 is pressed, the information in the corresponding row is deleted.

Then, when the registration button V57 is pressed, the inputted details are finalized and the inputted details are stored in the storage unit 35 and thus registered. Meanwhile, when the cancel button V58 is pressed, the screen returns to the menu screen ES1.

When the button F21, of the buttons F21, F22 arranged in the fixed display area F2 of the medication status registration screen ES5, is pressed, the screen shifts to the sleep registration screen ES6. When the button F22 is pressed, the screen shifts to the dietary composition registration screen ES4.

### [Configuration and Operation of Sleep Registration Screen]

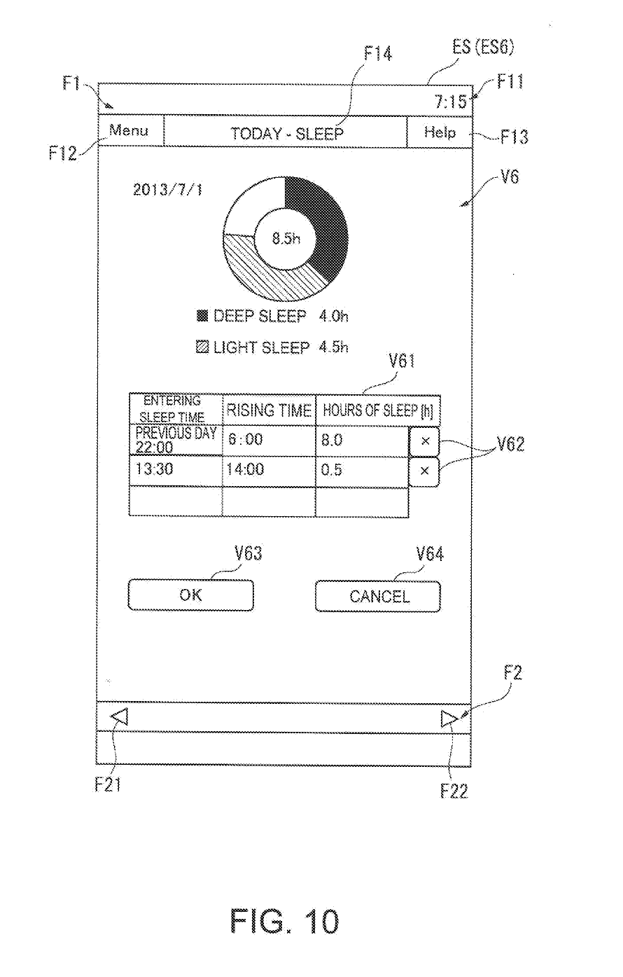

FIG. 10 is a view showing an example of the sleep registration screen ES6, of the execution screens ES.

When the selection item V15 is pressed on the menu screen ES 1 or when the button F22 is pressed on the medication status registration screen ES5, the sleep registration screen ES6 shown in FIG. 9 is displayed. This sleep registration screen ES6 is a screen for registering hours of sleep of the user, and includes the fixed display areas F1, F2 and a variable display area V6.

The date of displayed data is arranged at the top of the variable display area V6, and hours of light sleep and deep sleep, respectively, of the user, and a pie chart showing the ratio of hours of each sleep are arranged substantially in the middle.

At the center of the variable display area V6, a table V61 is arranged in which the start time and end time of sleep and hours of sleep that are already registered are set. As these items of information, each time and hours may be inputted in a row where data is not set in this table V61, or may be automatically set in each row on the basis of action information received from the measuring device 2. Also, by pressing a delete button V62 arranged on the right side in each row in the table V61, the data in the corresponding row can be deleted.

Then, when a registration button V63 arranged at the bottom of the variable display area V6 is pressed, the information newly inputted to the table V61 is stored in the storage unit 35 and thus registered. Meanwhile, when a cancel button V64, similarly arranged at the bottom, is pressed, the screen shifts to the menu screen ES1.

When the button F21, of the buttons F21, F22 arranged in the fixed display area F2 of the sleep registration screen ES6, is pressed, the screen shifts to the medication status registration screen ES5. When the button F22 is pressed, the screen shifts to the exercise display screen ES2.

### [Configuration and Operation of Calendar Display Screen]

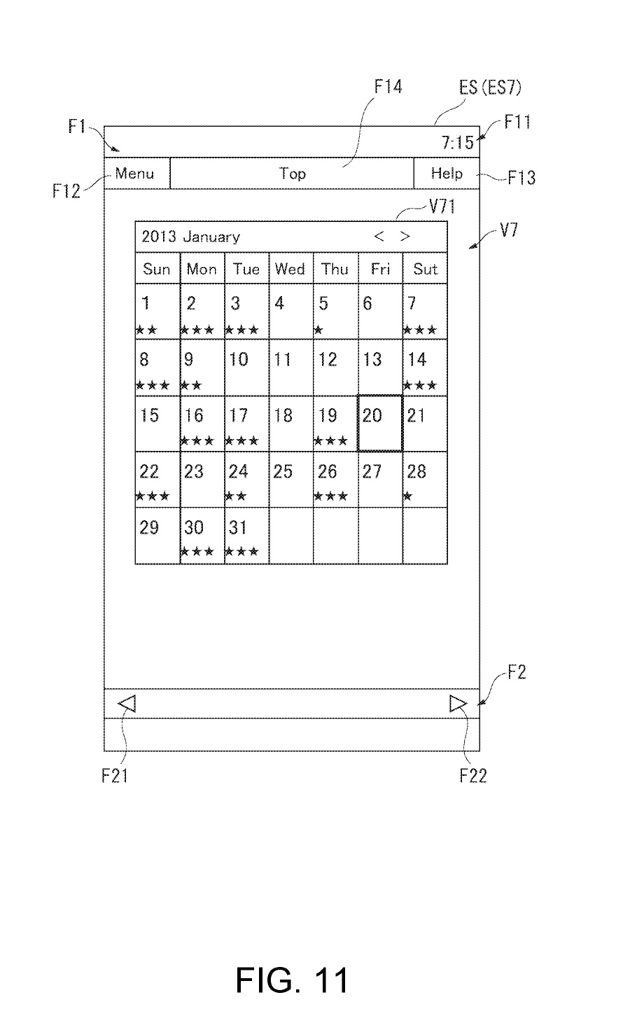

FIG. 11 is a view showing an example of the calendar display screen ES7, of the execution screens ES.

When the button V17 arranged in the menu screen ES1 is pressed, the calendar display screen ES7 shown in FIG. 11 is displayed. This calendar display screen ES7 includes the fixed display areas F1, F2 and a variable display area V7.

A calendar V71 of the month in question is arranged in the variable display area V7, and the degree of achievement of the target exercise and the target meal volume is indicated by star marks in each date display section in the calendar V71. When these date display sections are pressed, the screen shifts to a screen where detailed data of the corresponding date can be viewed, or to the menu screen ES1 of the corresponding date, though not illustrated.

Every time the button F21, of the buttons F21, F22 arranged in the fixed display area F2 of the calendar display screen ES7, is pressed, the screen shifts to the calendar display screen ES7 of the previous month, whereas every time the button F22 is pressed, the screen shifts to the calendar display screen ES7 of the later month.

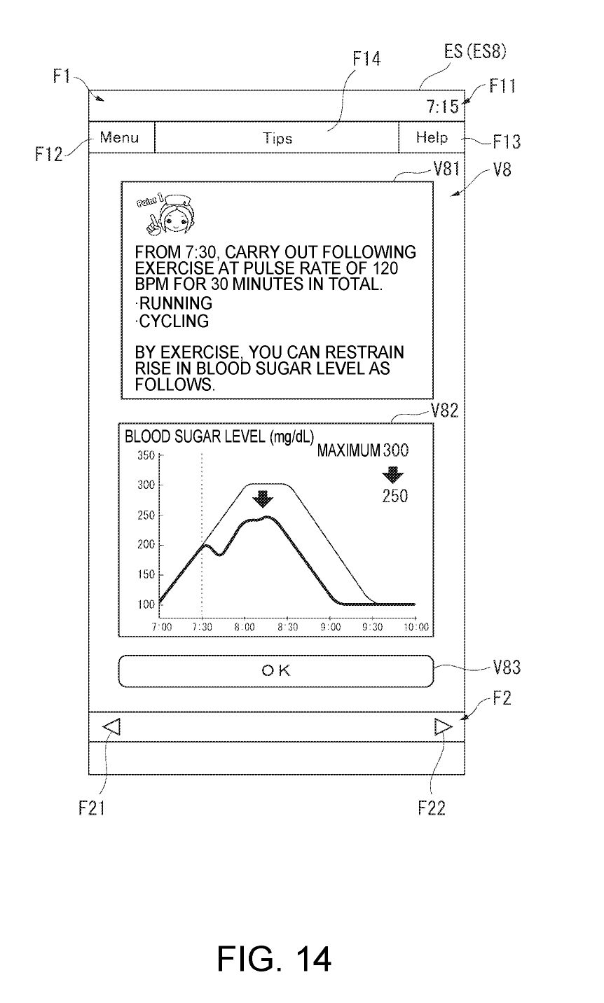

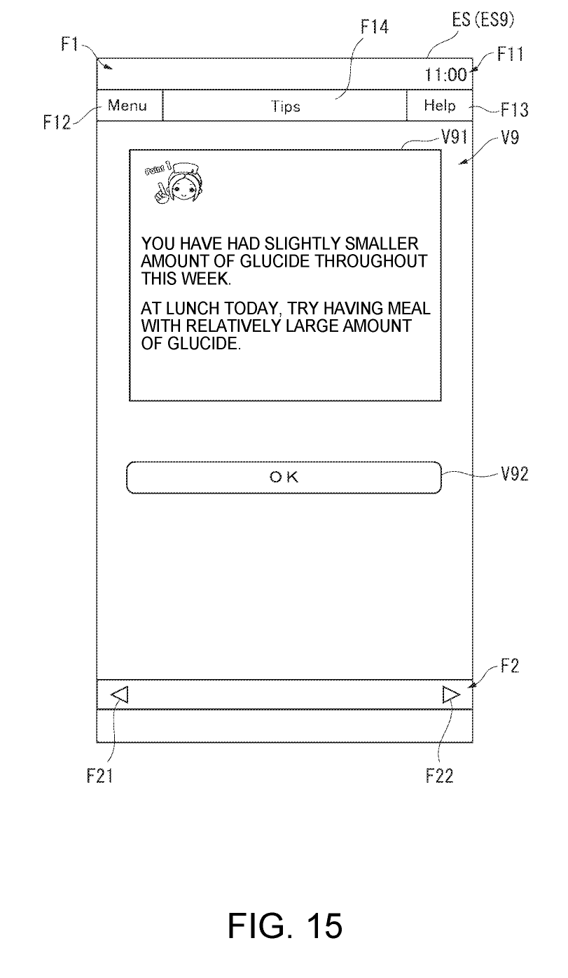

The above execution screens ES of the exercise support application are described as an example. Therefore, the layout of the input items and buttons or the like on each screen, and the details or the like of the arranged input items and selection items are not limited to the above description and can be changed when appropriate. The same applies to the layouts and the details of the execution screens ES shown in FIGS. 14 and 15 below (exercise suggestion screen ES8 and information suggestion screen ES9).

### [Configuration of Application Execution Unit]

Back to FIG. 3, the application execution unit 37B has an information registration unit 375, a timing setting unit 376, an exercise setting unit 377, an exercise correction unit 378, an exercise type extraction unit 379, a blood sugar level prediction unit 380, and a suggestion information generation unit 381, as functional units implemented by executing the exercise support application.

### [Configuration of Information Registration Unit]

The information registration unit 375 causes the information of the respective items inputted or selected by the user on the respective screens ES2 to ES6 and ES4A to be stored in the storage unit 35 in association with the dates displayed on the respective screens ES2 to ES6 and ES4A, thus registering the information of the respective items.

### [Configuration of Timing Setting Unit]

The timing setting unit 376, the exercise setting unit 377, the exercise correction unit 378, the exercise type extraction unit 379, the blood sugar level prediction unit 380 and the suggestion information generation unit 381 function when executing the exercise suggestion processing in which an exercise corresponding to the dietary composition of the user is suggested.

The timing setting unit 376 sets a start timing of the exercise to be carried out by the user. This timing setting unit 376 sets the start timing on the basis of blood sugar level change information indicating the way the blood sugar level of the user rises (ascent curve), stored in the storage unit 35 in advance.

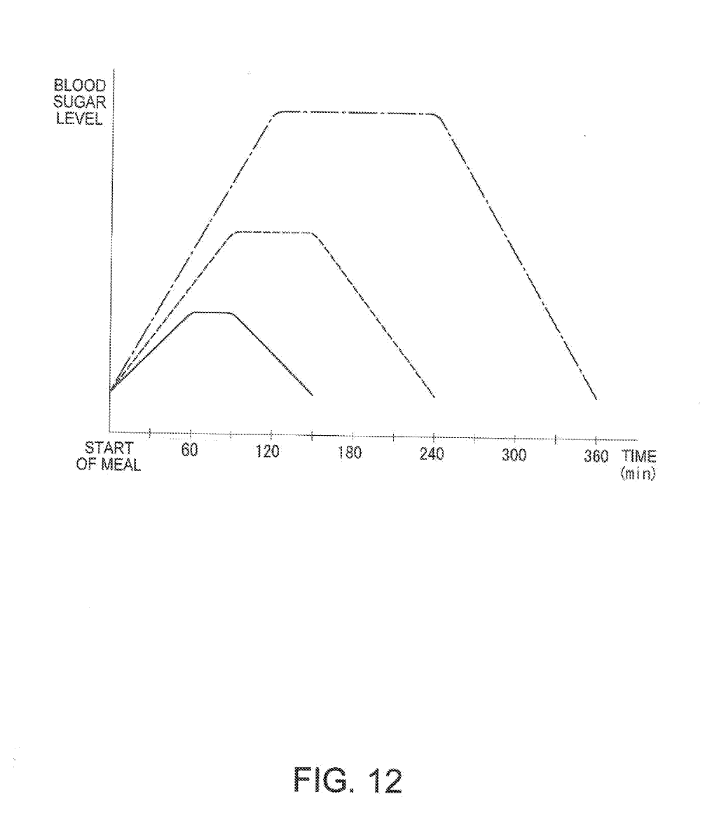

FIG. 12 is a view showing an example of changes in the blood sugar level according to the degree of progression of diabetes.

Here, in general, the blood sugar level rises from the start of a meal and reaches a maximum blood sugar level, and then drops after a state of no fluctuation continues for a while. Such changes in the blood sugar level vary depending on the degree of progression of diabetes, as shown in FIG. 12. That is, an ascent phase where the blood sugar level rises after a meal, a balanced phase where there is little change in the blood sugar level, and a descent phase where the blood sugar level drops vary depending on the degree of progression of diabetes.

For example, for a person likely to develop diabetes who has insulin secretion similar to that of a healthy person, the ascent phase is approximately 60 minutes, the balanced phase is approximately 30 minutes, and the descent phase is approximately 60 minutes, as indicated by a solid line in FIG. 12. Therefore, the blood sugar level 120 minutes after the start of a meal tends to be lower than the blood sugar level 60 minutes after the start of a meal.

Meanwhile, for a mild diabetic who has less insulin secretion than a healthy person, the ascent phase is approximately 90 minutes, the balanced phase is approximately 60 minutes, and the descent phase is approximately 90 minutes, as indicated by a dotted line in FIG. 12. Therefore, the blood sugar level 120 minutes after the start of a meal tends to be higher than the blood sugar level 60 minutes after the start of a meal.

Also, for a severe diabetic with very little insulin secretion, the ascent phase is approximately 120 minutes, the balanced phase is approximately 120 minutes, and the descent phase is approximately 120 minutes, as indicated by a dotted chain line in FIG. 12. Therefore, a state with a relatively high blood sugar level tends to continue, and the blood sugar level 120 minutes after the start of a meal tends to be higher than the blood sugar level 60 minutes after the start of a meal.

The way the blood sugar level rises in each user can be measured by OGTT, for example, and the blood sugar level change information indicating the way the blood sugar level rises is stored in the storage unit 35.

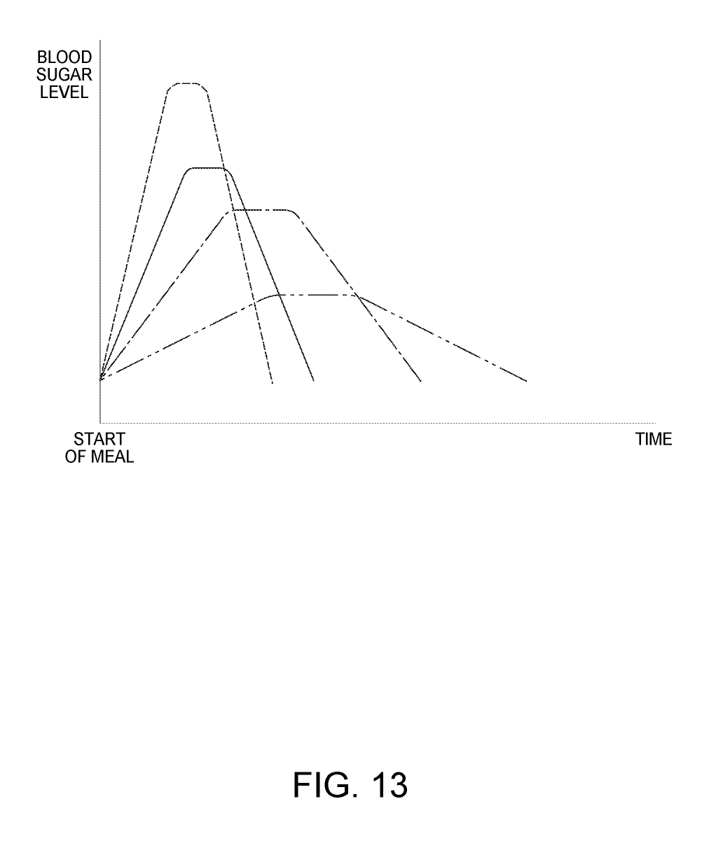

FIG. 13 is a view showing an example of changes in the blood sugar level according to the PFC ratio (component ratio of protein, fat and carbohydrate) in a meal.

Also, the duration of each of the ascent phase, the balanced phase and the descent phase also varies depending on the PFC ration in a meal, as shown in FIG. 13.

For example, when a meal indicated by a dotted line in FIG. 13 (a meal with 80% or more consisting of carbohydrate) is taken, the ascent phase, the balanced phase and the descent phase are approximately 0.8 times those in the case where a normal meal indicated by a solid line in FIG. 13 (a meal with 50% or more and 70% or less consisting of carbohydrate) is taken. Meanwhile, when a meal indicated by a dotted chain line in FIG. 13 (a meal with 30% or more consisting of protein) is taken, the ascent phase, the balanced phase and the descent phase are approximately 1.5 times those in the case where the normal meal is taken. When a meal indicated by a double-dotted chain line in FIG. 13 (a meal with 40% or more consisting of fat) is taken, the ascent phase, the balanced phase and the descent phase are approximately 2.0 times those in the case where the normal meal is taken.

It is thought that, with diabetes, blood vessels are more likely to be damaged when the blood sugar level fluctuate largely, thus causing various complications. Therefore, in order to reduce the range of fluctuation in the blood sugar level, that is, the maximum blood sugar level, a high-intensity exercise needs to be carried out during the period when the blood sugar level rises because of a meal, thus lowering the maximum blood sugar level. When the change in the blood sugar level is in the balanced phase after the maximum blood sugar level is reached, the blood sugar level can be lowered even by a moderate exercise. Moreover, during the descent of the blood sugar level, the blood sugar level can be easily lowered even with a relatively low-intensity exercise (mild exercise).

Considering these, the timing setting unit 376 in this embodiment sets a start timing of an exercise to the period when the blood sugar level of the user rises after a meal, on the basis of the blood sugar level change information and the dietary information.

Specifically, as the start timing, the timing setting unit 376 sets a timing within 60 minutes from the start of a meal for the person likely to develop diabetes, sets a timing within 90 minutes from the start of a meal for the mild diabetic, and sets a timing within 120 minutes after the start of a meal for the severe diabetic. Then, the timing setting unit 376 corrects (adjusts) the start timing on the basis of the PFC ratio (PFC ratio of the meal immediately before) included in the dietary information registered on the dietary composition registration screen ES4.

The start timing of an exercise thus set is stored in the storage unit 35 by the timing setting unit 376.

Although it might be considered preferable to exercise immediately after a meal, if an exercise is started within 30 minutes after a meal, physical conditions tend to become worse for reasons such as disturbed digestion. Therefore, it is preferable that the timing setting unit sets the start timing 30 minutes after a meal or later.

Meanwhile, if the blood sugar level change information of the user is not registered, the timing setting unit 376 may set, as the start timing of the exercise, the time 30 minutes after the start time of a meal, which is determined as the time when the blood sugar level of the user rising with the intake of the meal can be reduced, regardless of the degree of progression of diabetes.

### [Configuration of Exercise Setting Unit]

Back to FIG. 3, the exercise setting unit 377 sets an exercise to be carried out by the user. That is, the exercise setting unit 377 sets a target value of exercise information (target exercise information) including exercise intensity and exercise duration of this exercise. Specifically, the exercise setting unit 377 sets the target exercise information on the basis of the age of the user registered on an initial setting screen (not illustrated) of the application, and user information about the presence/absence of exercise habits and the like.

For example, if the patient is 40 years of age and has exercise habits, the exercise setting unit 377 sets 50% HRR (hear rate reserved) or a pulse rate of 120 bpm as the target exercise intensity and sets the target exercise duration of 30 minutes. Meanwhile, if the patient is 40 years of age but has no exercise habits, the exercise setting unit 377 sets 40% HRR or a pulse rate of 100 bpm as the target exercise intensity and sets the target exercise duration of 30 minutes.

Such target exercise information may be set by the user or may be set by a third party (for example, a medical practitioner such as a doctor) as well. In this case, the target exercise information may be set by the user or the third party operating the information processing device 3, or may be set by receiving data including the target exercise information from an external device.

Also, while the target exercise intensity in the above description is expressed by an exercise intensity based on the Karvonen method, the target exercise intensity may also be expressed by an exercise intensity such as METs, which is the amount of oxygen consumed, or based on the Borg scale (perceived exercise intensity).

### [Configuration of Exercise Correction Unit]

The exercise correction unit 378 corrects the exercise information set by the exercise setting unit 377 on the basis of the dietary composition in the past, and corrects (adjusts) the exercise to be carried out by the user, to a more effective exercise for the user.

Here, in general, if the amount of glucide in a meal is larger, the maximum blood sugar level after the intake of the meal tends to be higher.

Therefore, the exercise correction unit 378 acquires the amount of glucide in the meal from the dietary information indicating the composition of the most recent meal registered on the dietary composition registration screen ES4.

Then, if the amount of glucide is below a first threshold (for example, 50 g), the exercise correction unit 378 reduces the target exercise intensity. In this case, for example, if the target exercise intensity is 40% HRR, the exercise correction unit 378 subtracts 10% HRR to correct the target exercise intensity to 30% HRR. Alternatively, the exercise correction unit 378 reduces the exercise duration by 10%.

Meanwhile, if the amount of glucide is above a second threshold (for example, 100 g) higher than the first threshold, the exercise correction unit 378 increases the target exercise intensity. In this case, for example, if the target exercise intensity is 40% HRR, the exercise correction unit 378 adds 10% HRR to correct the target exercise intensity to 50% HRR. Alternatively, the exercise correction unit 378 extends the exercise duration by 10%.

Thus, an effective exercise intensity corresponding to the amount of glucide in the meal can be calculated.

Also, the ascent phase, the balanced phase and the descent phase of the blood sugar level after the intake of a meal vary, depending on the PFC ratio in the meal, as described above.

Therefore, the exercise correction unit 378 acquires the PFC ratio in the meal from the dietary information indicating the composition of the most recent meal. Then, if 40% or more of the meal consists of fat, the exercise correction unit 378 executes correction to reduce the target exercise intensity by 10%, for example. Meanwhile, if 30% or more of the meal consists of protein, the exercise correction unit 378 executes correction to reduce the target exercise intensity by 5%, for example. Moreover, if 80% or more of the meal consists of carbohydrate, the exercise correction unit 378 executes correction to increase the target exercise intensity by 5%, for example.

Thus, an effective exercise intensity corresponding to the PFC ratio in the meal can be calculated.

Such correction of the target exercise information according to the PFC ratio is executed in the case where the amount of glucide in the meal is 50 g or more. Also, the case where 40% or more of the meal consists of fat is given the top priority, and the case where 80% or more of the meal consists of carbohydrate has a low priority. Therefore, if 40% of the meal consists of fat and 30% consists of protein, the correction in the case of the meal with 40% consisting of fat is carried out.

Moreover, the exercise correction unit 378 may correct the target exercise duration on the basis of the total calories and meal volume of the most recent meal, included in the dietary information.

### [Configuration of Exercise Type Extraction Unit]

The exercise type extraction unit 379 extracts an exercise type corresponding to the target exercise intensity set by the exercise setting unit 377 and corrected by the exercise correction unit 378. Specifically, the exercise type extraction unit 379 acquires the target exercise intensity and acquires an exercise type corresponding to the target exercise intensity from a table stored in the storage unit 35. At this time, if there is a plurality of corresponding exercise types, the exercise type extraction unit 379 acquires all of the plurality of corresponding exercise types.

### [Configuration of Blood Sugar Level Prediction Unit]

The blood sugar level prediction unit 380 is equivalent to the post-meal blood sugar level prediction unit and the post-exercise blood sugar level prediction unit of the invention, and predicts changes in the blood sugar level of the user and generates a predicted blood sugar level change curve indicating the changes in the blood sugar level in time series. Specifically, the blood sugar level prediction unit 380 generates a predicted blood sugar level change curve (equivalent to the post-meal blood sugar level change) which predicts changes in the blood sugar level of the user after the most recent intake of a meal, on the basis of the blood sugar level and the most recent dietary information of the user registered on the execution screens ES, and the blood sugar level change information or the like. Also, the blood sugar level prediction unit 380 generates a predicted blood sugar level change curve (equivalent to the post-exercise blood sugar level change) which predicts changes in the blood sugar level of the user in the case where the exercise indicated by the target exercise information is carried out at the start timing set by the timing setting unit 376. In this case, the predicted blood sugar level change curve may be corrected on the basis of the insulin resistance and the amount of insulin secreted of the user, and the result of the OGTT test.

For the generation of such a predicted blood sugar level change curve, the method disclosed in JP-A-2011-104138 can be employed.

### [Configuration of Suggestion Information Generation Unit]

FIG. 14 is a view showing an example of the exercise suggestion screen ES8 in which suggestion information generated by the suggestion information generation unit 381 is incorporated, of the execution screens ES.

The suggestion information generation unit 381 generates suggestion information to be suggested to the user. Specifically, the suggestion information generation unit 381 generates suggestion information set on the exercise suggestion screen ES8 shown in FIG. 14.

This exercise suggestion screen ES8 is a screen for suggesting the start timing, the target exercise information and the exercise type corresponding to the target exercise intensity, to the user, and includes the fixed display areas F1, F2 and a variable display area V8.

In the variable display area V8, an exercise suggestion area V81, a predicted blood sugar level change suggestion area V82, and a confirmation button V83 are arranged.

In the exercise suggestion area V81, an explanation text including the start timing, the target exercise duration, and at least one of the target exercise intensity and the exercise type corresponding to the target exercise intensity, is set. On the exercise suggestion screen ES8 shown in FIG. 14, an explanation text which includes “7:30” as the start timing, “30 minutes” as the target exercise duration, “pulse rate 120 bpm” as the target exercise intensity, and “running” and “cycling” as the exercise type corresponding to the exercise intensity, is set. The suggestion information generation unit 381 generates such an explanation text.

When displaying the exercise suggestion screen ES8, the audio output unit 34 may output a sound corresponding to the explanation text set in the exercise suggestion area V81.

In the predicted blood sugar level change suggestion area V82, a graph including the predicted blood sugar level change curve generated by the blood sugar level prediction unit 380 is set. Specifically, the graph set in the predicted blood sugar level change suggestion area V82 includes a predicted blood sugar level change curve in the case where the suggested exercise is not carried out (predicted blood sugar level change curve indicated by a thin line in FIG. 14) and a predicted blood sugar level change curve in the case where this exercise is carried out (predicted blood sugar level change curve indicated by a bold line in FIG. 14), and also includes a dotted line indicating the start timing. Moreover, in the blank area of the predicted blood sugar level change suggestion area V82, predicted maximum blood sugar levels in the case where the exercise is not carried out and in the case where the exercise is carried out are set. Also, in the predicted blood sugar level change suggestion area V82, an indication emphasizing the graph and the maximum blood sugar level that will be changed by carrying out the exercise (in this embodiment, an arrow) is set. Such setting details in the area V82 are generated by the suggestion information generation unit 381.

When the confirmation button V83 is pressed on such an exercise suggestion screen ES8, the screen returns to the screen before the shift to the exercise suggestion screen ES8.

FIG. 15 is a view showing an example of the information suggestion screen ES9, of the execution screens ES.

The suggestion information generation unit 381 suitably generates suggestion information to be suggested to the user, according to the current time measured by the clock unit 374. Then, the display control unit 372 causes the display unit 33 to display the information suggestion screen ES9 including this suggestion information.

This information suggestion screen ES9 includes the fixed display areas F1, F2 and a variable display area V9, as shown in FIG. 15. In the variable display area V9, an information suggestion area V91 where the suggestion information generated by the suggestion information generation unit 381 is set, and a confirmation button V92 similar to the confirmation button V83, are arranged.

Then, the suggestion information generation unit 381 generates suggestion information based on the dietary information that is already registered, for example. For example, on the basis of the dietary information corresponding to a predetermined period, the suggestion information generation unit 381 determines the PFC ratio corresponding to this period, and generates suggestion information that gives a warning set in the information suggestion area V91 of the information suggestion screen ES9 shown in FIG. 14, when the PFC ratio lacks balance and the time assumed to be pre-meal is reached. Presenting the information suggestion screen ES9 where such suggestion information is set to the user can be an opportunity to improve the dietary habits of the user.

Also, the suggestion information generation unit 381 generates suggestion information that offers advice corresponding to the current time, and causes the information suggestion screen ES9 with this suggestion information set, to be displayed. For example, such suggestion information can be suggestion information generated before bedtime and including a point that “Since a state of high blood sugar level tends to be maintained during sleep due to the meal before bedtime, reduce your blood sugar level sufficiently by exercise or the like before going to bed”.

Similarly, the suggestion information generation unit 381 generates suggestion that offers advice on the meal on the basis of the current time, and causes the information suggestion screen ES9 with this suggestion information set, to be displayed.

Moreover, the suggestion information generation unit 381 determines whether the user has been taking an oral medicine or insulin administration or not, that is, whether the user has been carrying out a pharmacotherapy or not, on the basis of the information registered on the medication status registration screen ES5. Then, if it is determined that the user has been carrying out a pharmacotherapy, the suggestion information generation unit 381 generates suggestion information that offers advice for a person subject to the pharmacotherapy after displaying the exercise suggestion screen ES8, and causes the information suggestion screen ES9 with this suggestion information set (not illustrated), to be displayed. Thus, the advice for a person subject to the pharmacotherapy can be presented.

As with the exercise suggestion screen ES8, the audio output unit 34 may output a sound corresponding to the suggestion information set on the information suggestion screen ES9. Moreover, a configuration to display information recommending a specific dietary menu in the variable display area V9 may be employed.

### [Exercise Suggestion Processing]

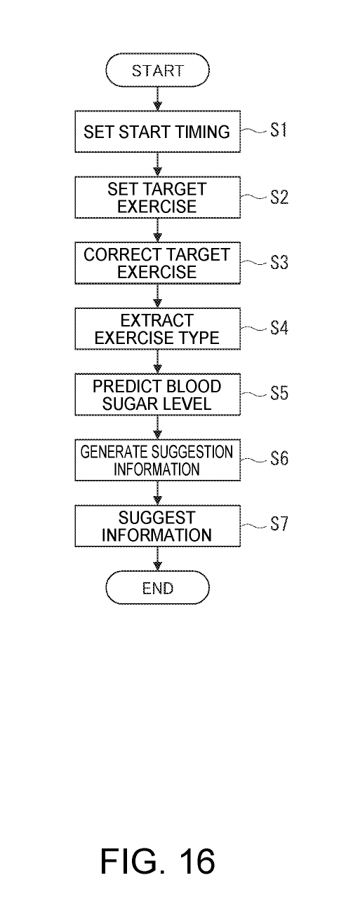

FIG. 16 is a flowchart showing the exercise suggestion processing executed by the control unit 37.

The exercise suggestion processing is processing executed on the basis of the exercise suggestion program included in the exercise support application. The execution of the exercise suggestion processing is on standby while the exercise support application is executed. Then, the exercise suggestion processing is executed by the control unit 37, for example, when the user registers a dietary composition on the dietary composition registration screen ES4 or when the user carries out an input operation to execute the exercise suggestion processing.

In this exercise suggestion processing, first, the timing setting unit 376 sets the start timing at which the user is made to start the exercise (Step S1), as shown in FIG. 16.

Also, the exercise setting unit 377 sets target exercise information (target exercise intensity and target exercise duration) on the basis of the user information (Step S2). Alternatively, in this Step S2, the exercise setting unit 377 acquires preset target exercise information.

After this Step S2, the exercise correction unit 378 corrects the target exercise information on the basis of the most recent dietary information or the like (Step S3).

Next, the exercise type extraction unit 379 extracts an exercise type corresponding to the target exercise intensity included in the target exercise information, from the table stored in the storage unit 35 (Step S4).

Also, the blood sugar level prediction unit 380 generates a predicted blood sugar level change curve in the case where the exercise indicated by the target exercise information is not carried out, and a predicted blood sugar level change curve in the case where this exercise is carried out (Step S5).

Then, the suggestion information generation unit 381 generates suggestion information to be suggested to the user, that is, suggestion information including the start timing, the target exercise information, the exercise type and the respective predicted blood sugar level change curves (Step S6).

After this Step S6, the display control unit 372 causes the drawing unit 36 to draw the exercise suggestion screen ES8 including this suggestion information, and causes the display unit 33 to display this exercise suggestion screen ES8 (Step S7).

With this, the exercise suggestion processing ends.

### Advantageous Effects of Embodiment

The exercise support system 1 according to the embodiment described above has the following advantageous effects.

The target exercise information set by the exercise setting unit 377 on the basis of the user information is corrected by the exercise correction unit 378 on the basis of the dietary information indicating the meal which the user has had. Then, the exercise suggestion screen ES8 including the target exercise duration and the target exercise intensity included in the target exercise information, and the exercise type corresponding to the target exercise intensity, is displayed on the display unit 33, and details of the exercise to be carried out are thus suggested to the user. Thus, an exercise corresponding to the dietary composition of the user can be suggested to the user. Therefore, for example, an exercise that can effectively retrain a rise in the blood sugar level after the meal can be suggested to the user.

If the amount of glucide in the meal is large, the maximum blood sugar level after the meal tends to be higher than in the case where the amount of glucide is small. In this regard, the exercise correction unit 378 corrects the target exercise information according to the amount of glucide in the meal. Thus, an exercise which can effectively restrain a rise in the blood sugar level after the meal can be securely suggested to the user.

The way the blood sugar level rises changes according to the PFC ratio in the meal. In this respect, the exercise correction unit 378 corrects the target exercise information on the basis of the PFC ratio in the meal. Thus, an exercise which can more effectively restrain a rise in the blood sugar level after the meal can be securely suggested to the user.

The timing setting unit 376 sets the start timing at which the user is made to start the suggested exercise, within a period when the blood sugar level rises after a meal. Thus, since the maximum blood sugar level can be lowered as the user carries out the suggested exercise, the range of fluctuation in the blood sugar level can be reduced. Therefore, damage to blood vessels can be restrained, and improvement in insulin resistance is expected by executing the exercise repeatedly.

The exercise suggestion screen ES8 displayed on the display unit 33 includes the predicted blood sugar level change curve generated by the blood sugar level prediction unit 380 and corresponding to the case where the exercise indicated by the suggested target exercise information is carried out. Thus, the user can grasp how the blood sugar level changes by carrying out the suggested exercise. Therefore, the user's motivation for the exercise can be enhanced.

The exercise suggestion screen ES8 includes not only the predicted blood sugar level change curve in the case where the exercise is carried out, but also the predicted blood sugar level change curve in the case where the exercise is not carried out. Thus, the user can compare the case where the suggested exercise is carried out with the case where the exercise is not carried out. In this case, since these change curves are shown in one graph, the respective change curves can be easily compared. Therefore, the user's motivation for the exercise can be enhanced further.

### Modifications of Embodiment

The invention is not limited to the above embodiment and includes modifications, improvements and the like within a range that can achieve the objective of the invention.

In the embodiment, the information processing device 3 receives action information or the like from the measuring device 2 and stores the information in the storage unit 35. However, the invention is not limited to this. That is, the measuring device 2 is not necessarily needed and the information processing device 3 can operate independently (single-handedly). Also, a part or all of the functions and configurations of the information processing device 3 may be incorporated in the measuring device 2 to implement the invention. Moreover, instead of introducing the exercise support application as an application in the information processing device 3, a configuration that functions when executing the exercise support application may be incorporated in advance in the information processing device 3 or the measuring device 2 formed as a mobile terminal.

The numerical values given in the embodiment are numerical values given as examples and can be changed when appropriate.

In the embodiment, the exercise setting unit 377 sets the target exercise intensity and the target exercise duration on the basis of the user information, and the exercise correction unit 378 corrects the target exercise intensity on the basis of the most recent dietary information or the like. However, the invention is not limited to this. That is, the exercise correction unit 378 may correct the target exercise duration instead of or in addition to the target exercise intensity. Moreover, the exercise correction unit 378 may correct at least one of the target exercise duration and the target exercise intensity, not only on the basis of the most recent dietary information but also on the basis of the cumulative amount of glucide in a predetermined period and the PFC ratio in a predetermined period. Furthermore, the exercise correction unit 378 may correct at least one of the target exercise duration and the target exercise intensity on the basis of other information included in the dietary information, for example, on the basis of the calories of the meal. In addition, the suggested information may be only one of the exercise intensity and the exercise duration.

In the embodiment, the timing setting unit 376 sets the start timing of the exercise in the ascent phase where the blood sugar level rises after a meal. However, the invention is not limited to this. For example, the start timing may be set in the balanced phase and the descent phase. Then, if the start timing is set in the balanced phase and the descent phase, the exercise correction unit 378 may correct at least one of the target exercise intensity and the target exercise duration on the basis of the set start timing.

In the embodiment, the exercise setting unit 377 sets the target exercise duration and the target exercise intensity on the basis of the age of the user and the presence/absence of exercise habits included in the user information. However, the invention is not limited to this. For example, the target exercise duration and the target exercise intensity may be set on the basis of other information. Moreover, the frequency of the exercise to be carried out by the user (for example, once every three days or more) may be set on the basis of the presence/absence of exercise habits, and this frequency may be suggested to the user.

In the embodiment, the exercise suggestion screen ES8 includes a predicted blood sugar level change curve indicating changes in the blood sugar level in the case where an exercise is carried out, and also a predicted blood sugar level change curve indicating changes in the blood sugar level in the case where the exercise is not carried out. However, the invention is not limited. For example, the exercise suggestion screen ES8 may include only one of these predicted blood sugar level change curves or may include none.

Also, the exercise suggestion screen ES8 includes the exercise duration of the exercise to be carried out by the user, and also the exercise intensity and the exercise type corresponding to this exercise intensity. However, the invention is not limited to this. For example, it suffices that one of the exercise intensity and the exercise type is included. The suggested exercise intensity may be indicated in other forms of exercise intensity as well.

In the embodiment, the exercise suggestion program is included in the exercise support application. However, the invention is not limited to this. That is, the exercise suggestion program may be executed independently.

In the embodiment, the exercise suggestion program is stored in the storage unit 35. However, the invention is not limited to this. For example, the exercise suggestion program may be stored in a server on a network, or on a disk-type recording medium and an external semiconductor memory or the like, and may be acquired from each when executing the exercise suggestion program. That is, the saving format and provision format of the exercise suggestion program may be any format as long as the exercise suggestion program can be utilized by a computer such as the information processing device 3.

### REFERENCE SIGNS LIST

3 . . . information processing device (exercise suggestion device), 33 . . . display unit (suggestion unit), 34 . . . audio output unit (suggestion unit), 376 . . . timing setting unit, 378 . . . exercise correction unit, 380 . . . blood sugar level prediction unit (post-exercise blood sugar level prediction unit, post-meal blood sugar level prediction unit).

1. An exercise suggestion device comprising:
an exercise correction unit which corrects exercise information set on the basis of user information about a user and including exercise duration and exercise intensity of an exercise to be carried out by the user, on the basis of dietary information indicating composition of a meal of the user; and
a suggestion unit which suggests at least one of the exercise intensity included in the exercise information that is corrected and an exercise type corresponding to the exercise intensity, and the exercise duration included in the exercise information that is corrected.

2. The exercise suggestion device according to claim 1, wherein
the exercise correction unit corrects the exercise information on the basis of an amount of glucide in the meal included in the dietary information.

3. The exercise suggestion device according to claim 1, wherein
the exercise correction unit corrects the exercise information on the basis of a ratio of protein, fat and carbohydrate in the meal included in the dietary information.

4. The exercise suggestion device according to claim 1, comprising
a timing setting unit which sets a start timing of the exercise on the basis of blood sugar level change information indicating the way the blood sugar level of the user included in the user information rises,
wherein the suggestion unit suggests the start timing.

5. The exercise suggestion device according to claim 1, comprising
a post-exercise blood sugar level prediction unit which predicts a post-exercise blood sugar level change that is a change in the blood sugar level of the user in the case where the exercise indicated by the exercise information is carried out,
wherein the suggestion unit suggests the post-exercise blood sugar level change.

6. The exercise suggestion device according to claim 5, comprising
a post-meal blood sugar level prediction unit which predicts a post-meal blood sugar level change that is a change in the blood sugar level of the user in the case where the meal is taken, on the basis of the dietary information,
wherein the suggestion unit suggests the post-meal blood sugar level change and the post-exercise blood sugar level change.

7. An exercise suggestion system comprising:
an exercise information generation unit which generates exercise information including exercise duration and exercise intensity on the basis of user information about a user;
a dietary information acquisition unit which acquires dietary information indicating composition of a meal of the user;
an exercise correction unit which corrects the exercise information on the basis of the dietary information; and
a suggestion unit which suggests at least one of an exercise type corresponding to the exercise information that is corrected and the exercise intensity that is corrected, and the exercise duration that is corrected.

8. The exercise suggestion system according to claim 7, wherein
the exercise correction unit corrects the exercise information on the basis of an amount of glucide in the meal included in the dietary information.

9. The exercise suggestion system according to claim 7, wherein
the exercise correction unit corrects the exercise information on the basis of a ratio of protein, fat and carbohydrate in the meal included in the dietary information.

10. The exercise suggestion system according to claim 7, comprising
a timing setting unit which sets a start timing at which an exercise corresponding to the exercise information corrected by the exercise correction unit is started, on the basis of the user information,
wherein the suggestion unit suggests the start timing.

11. The exercise suggestion system according to claim 10, wherein
the user information includes blood sugar level change information indicating the way the blood sugar level of the user rises.

12. The exercise suggestion system according to claim 10, wherein
the start timing is information indicating at least one of a time at which the exercise is started and a time period until the exercise is started.

13. An exercise suggestion server comprising:
an exercise information generation unit which generates exercise information including exercise duration and exercise intensity on the basis of user information about a user;
a dietary information acquisition unit which acquires dietary information indicating composition of a meal of the user;
an exercise correction unit which corrects the exercise information on the basis of the dietary information; and
a communication unit which communicates at least one of an exercise type corresponding to the exercise information that is corrected and the exercise intensity that is corrected, and the exercise duration that is corrected.

14. The exercise suggestion server according to claim 13, wherein
the exercise correction unit corrects the exercise information on the basis of an amount of glucide in the meal included in the dietary information.

15. The exercise suggestion server according to claim 13, wherein
the exercise correction unit corrects the exercise information on the basis of a ratio of protein, fat and carbohydrate in the meal included in the dietary information.

16. The exercise suggestion server according to claim 13, comprising
a timing setting unit which sets a start timing at which an exercise corresponding to the exercise information corrected by the exercise correction unit is started, on the basis of the user information,
wherein the suggestion unit suggests the start timing.

17. The exercise suggestion server according to claim 16, wherein
the user information includes blood sugar level change information indicating the way the blood sugar level of the user rises.

18. The exercise suggestion server according to claim 16, wherein
the start timing is information indicating at least one of a time at which the exercise is started and a time period until the exercise is started.

19. The exercise suggestion device according to claim 2, wherein
the exercise correction unit corrects the exercise information on the basis of a ratio of protein, fat and carbohydrate in the meal included in the dietary information.

20. The exercise suggestion device according to claim 19, comprising
a timing setting unit which sets a start timing of the exercise on the basis of blood sugar level change information indicating the way the blood sugar level of the user included in the user information rises,
wherein the suggestion unit suggests the start timing.

21. The exercise suggestion device according to claim 20, comprising
a post-exercise blood sugar level prediction unit which predicts a post-exercise blood sugar level change that is a change in the blood sugar level of the user in the case where the exercise indicated by the exercise information is carried out,
wherein the suggestion unit suggests the post-exercise blood sugar level change.

22. The exercise suggestion device according to claim 2, comprising
a timing setting unit which sets a start timing of the exercise on the basis of blood sugar level change information indicating the way the blood sugar level of the user included in the user information rises,
wherein the suggestion unit suggests the start timing.

23. The exercise suggestion device according to claim 3, comprising
a timing setting unit which sets a start timing of the exercise on the basis of blood sugar level change information indicating the way the blood sugar level of the user included in the user information rises,
wherein the suggestion unit suggests the start timing.

24. The exercise suggestion device according to claim 2, comprising
a post-exercise blood sugar level prediction unit which predicts a post-exercise blood sugar level change that is a change in the blood sugar level of the user in the case where the exercise indicated by the exercise information is carried out,
wherein the suggestion unit suggests the post-exercise blood sugar level change.

25. The exercise suggestion device according to claim 3, comprising
a post-exercise blood sugar level prediction unit which predicts a post-exercise blood sugar level change that is a change in the blood sugar level of the user in the case where the exercise indicated by the exercise information is carried out,
wherein the suggestion unit suggests the post-exercise blood sugar level change.

26. The exercise suggestion device according to claim 4, comprising
a post-exercise blood sugar level prediction unit which predicts a post-exercise blood sugar level change that is a change in the blood sugar level of the user in the case where the exercise indicated by the exercise information is carried out,
wherein the suggestion unit suggests the post-exercise blood sugar level change.
